(19) United States
(12) Patent Application Publication
(10) Pub. No.: US 2011/0268775 A1
(43) Pub. Date: Nov. 3, 2011

(54) NANOPARTICLE PHARMACEUTICAL FORMULATIONS

(75) Inventors: Kris Holt, Honeoye Falls, NY (US); Deepak Thassu, Fairpoint, NY (US); Michael R. Violante, Pittsford, NY (US)

(73) Assignee: PHARMANOVA, INC., Victor, NY (US)

(21) Appl. No.: 13/143,375

(22) PCT Filed: Jan. 5, 2010
(86) PCT No.: PCT/US2010/020109

§ 371 Date: Jul. 6, 2011

Related U.S. Application Data
(60) Provisional application No. 61/142,798, filed on Jan. 6, 2009.

(51) Int. Cl. A61K 9/14; A61K 31/196; A61K 31/4184; A61K 31/522; A61K 31/517; A61P 9/00; A61P 9/12; A61P 33/00; A61P 31/22; A61P 29/00; A61P 43/00; A61P 37/06; A61K 47/28; A61K 38/13; B82Y 5/00

(52) U.S. Cl. 424/400; 514/20.5; 514/567; 514/395; 514/263.38; 514/266.3; 514/772; 977/773

(57) ABSTRACT

The present invention is directed to methods of preparing nanoparticles of aqueous-insoluble compounds, particularly aqueous-insoluble bioactive (drug) compounds, and to compositions and medicaments obtained by these methods. These methods, compositions, and other inventive aspects of the present invention are based particularly on the use of bile acid compound(s) to prepare nanoparticles of aqueous-insoluble compounds.

### FIELD OF THE INVENTION

The present invention is directed to methods of preparing nanoparticles of aqueous-insoluble compounds, particularly aqueous-insoluble bioactive (drug) compounds, and to compositions and medicaments obtained by these methods. These methods, compositions, and other inventive aspects of the present invention are based particularly on the use of bile acid compound(s) to prepare nanoparticles of aqueous-insoluble compounds.

### BACKGROUND

The information provided below is not admitted to be prior art to the present invention, but is provided solely to assist the understanding of the reader.

Although many bioactive (drug) compounds are readily dissolved in water, a large number of bioactives have poor aqueous solubility, i.e., are, to varying extents, “aqueous-insoluble” (synonymously, “water-insoluble”). Such insolubility creates a variety of significant barriers to the effective use of such compounds, including difficulties in formulating such compounds for administration (e.g., when poor solubility results in difficulties in preparing solutions of drugs for injection or other routes of administration) and difficulties in ensuring that such compounds are effectively and rapidly released in the body even when effective administration is achieved. The consequences of such difficulties in administration and bioavailability can include drastic reduction in the effectiveness of promising drugs; therefore, numerous attempts to overcome these limitations of aqueous-insoluble drugs have been made.

Since the ability of aqueous-insoluble compounds to dissolve in water is generally inversely related to the average particle size of such compounds, an ability to formulate such compounds as very small (microscopic) particles generally results in the improved solubility of such compounds. Consequently, a variety of methods have been developed to produce aqueous-insoluble drug compounds in such particulated form, and particularly particles with diameters in the micron or sub-micron (nanometer) diameter range, i.e., generically, “nanoparticles.” These nanoparticles may be used in various forms to treat disease, including particularly as a component or components of a “formulation” comprising such nanoparticles.

Solvent/Anti-Solvent Processes. One methodology for producing nanoparticles of aqueous-insoluble drugs involves solvent/anti-solvent processes. Thus, for example, U.S. Pat. No. 4,826,689 to Violante et al. (but, Applicants note, incorrectly naming “Violanto” as one of the inventors), the disclosure of which is incorporated herein by reference in its entirety, discloses solvent/anti-solvent methods for making uniformly sized particles from aqueous-insoluble drugs or other organic compounds. First, an aqueous-insoluble organic compound is dissolved in a first (organic) water miscible solvent. Optionally, the solution is diluted with a non-solvent. Then, an aqueous precipitating second solvent is infused, precipitating non-aggregated particles with substantially uniform mean diameter. The particles are then separated from the organic solvent present in the mixture. Depending on the organic compound and the desired particle size, the parameters of temperature, ratio of non-solvent to organic solvent, infusion rate, stir rate, and volume can be varied according to the invention. The '689 patent discloses utilizing crystallization inhibitors (e.g., polyvinyl pyrrolidone) and surface-active agents/surfactants (e.g., poly(oxyethylene)-co-oxypropylene) to render the precipitate stable enough to be isolated by centrifugation, membrane filtration or reverse osmosis. See also U.S. Pat. No. 4,997,454 to the same inventors, the contents of which is also incorporated in its entirety by reference.

As another example, U.S. Pat. No. 5,118,528 to Fessi et al., the disclosure of which is incorporated herein by reference in its entirety, discloses a process for the preparation of dispersible colloidal systems of a substance in the form of spherical particles of the matrix type and of a size less than 500 nm (nanoparticles), comprising: combining (1) a first liquid phase consisting essentially of a solution of a film-forming material and a biologically active substance in a solvent or in a mixture of solvents to which may be added one or more surfactants, and (2) a greater volume of a second liquid phase consisting essentially of a non-solvent or a mixture of non-solvents for the film-forming material and biologically active substance and to which may be added one or more surfactants, the non-solvent or the mixture of non-solvents for the film-forming material and biologically active substance being miscible in all proportions with the solvent or mixture of solvents for the film-forming material and biologically active substance; thereby substantially instantaneously to precipitate from said solvent and said non-solvent composite particles of said film-forming material and biologically active substance to produce a colloidal suspension of composite nanoparticles. The '528 patent discloses that it produces particles of the substance smaller than 500 nm without the supply of energy. In particular the '528 patent states that it is undesirable to use high-energy equipment such as sonicators and homogenizers.

In a third example of a solvent/anti-solvent process, U.S. Pat. No. 6,607,784 to Kipp et al., the disclosure of which is incorporated herein by reference in its entirety, describes a similar process, but further including the additional step of adding energy to the to system. The invention provides a method for preparing submicron sized particles of an organic compound, the solubility of which is greater in a water-miscible first solvent than in a second solvent which is aqueous, the process including the steps of (i) dissolving the organic compound in the water-miscible first solvent to form a solution, (ii) mixing the solution with the second solvent to define a pre-suspension; and (iii) adding energy to the pre-suspension to form particles having an average effective particle size of 400 nm to 2 microns.

Nanoparticles Via Supercritical Fluid Processes. Another methodology for producing nanoparticles of aqueous-insoluble drugs involves supercritical fluid processes. Thus, for example, U.S. Pat. No. 5,360,478 to Krukonis et al., the disclosure of which is incorporated herein by reference in its entirety, discloses using a supercritical fluid (a gas or liquid at conditions of pressure and temperature above its critical point) or a gas at conditions near its vapor pressure to dissolve in and expand an organic liquid containing a dissolved solute. If the gas is not a solvent for the solute, i.e., the solid is substantially insoluble in the gas, the solid will crystallize when the organic liquid is sufficiently expanded.

Nanoparticles Via Milling Processes. A third exemplary methodology for producing nanoparticles of aqueous-insoluble drugs involves the milling down of larger particles to produce smaller particles. Thus, for example, U.S. Pat. No. 5,145,684 to Liversidge et al., the disclosure of which is incorporated herein by reference in its entirety, describes a wet milling process for preparing dispersible particles consisting essentially of a crystalline drug substance having a surface modifier adsorbed on the surface thereof in an amount sufficient to maintain an effective average particle size of less than about 400 nm, methods for the preparation of such particles and dispersions containing the particles. Pharmaceutical compositions containing the particles are useful in methods of treating mammals.

In another example of producing nanoparticles of aqueous-insoluble drugs by milling, U.S. Pat. No. 5,858,410 to Muller et al., the disclosure of which is incorporated herein by reference in its entirety, discloses a drug carrier system, prepared using a high amount of energy, comprising particles of at least one pure active compound which is insoluble, only sparingly soluble or moderately soluble in water, aqueous media and/or organic solvents, wherein said active ingredient is solid at room temperature and has an average diameter, determined by photon correlation spectroscopy (PCS) of 10 nm to 1,000 nm, and, when introduced into water, aqueous media and/or organic solvents, the active compound has an increased saturation solubility and an increased rate of dissolution compared with powders of the active compound prepared without using a high amount of energy.

In a third example of producing nanoparticles by milling, U.S. Pat. No. 5,510,118 to Bosch et al., the disclosure of which is incorporated herein by reference in its entirety, describes a process of preparing nanoparticulate drug substances comprising the steps of: preparing a premix of the drug substance and a surface modifier, and subjecting the premix to mechanical means such as a microfluidizer to reduce the particle size of the drug substance, the mechanical means producing shear, impact, cavitation and attrition.

Despite the availability of various techniques such as those described above that allow for the preparation of nanoparticles of aqueous-insoluble pharmaceutical compounds, there remains a need for better methods for making such nanoparticles. The present invention is responsive to this need, unmet in the prior art.

### SUMMARY OF THE INVENTION

The present invention is directed to methods of preparing nanoparticles of aqueous-insoluble compounds, particularly aqueous-insoluble bioactive (drug) compounds, and to compositions and medicaments obtained by these methods. These methods, compositions, and other inventive aspects of the present invention are based particularly on the use of bile acid compound(s) to prepare nanoparticles of aqueous-insoluble compounds.

In embodiment 1, the present invention is directed to a composition comprising nanoparticles, the nanoparticles comprising at least one aqueous-insoluble compound and at least one bile acid compound, where the at least one aqueous-insoluble compound represents at least 76% of the total weight of the combination of the at least one aqueous-insoluble compound plus the at least one bile acid compound in the nanoparticles.

In embodiment 2, the present invention is directed to the composition of embodiment 1, where the at least one bile acid compound represents less than 10% of the total weight of the combination of the at least one aqueous-insoluble compound plus the at least one bile acid compound in the nanoparticles.

In embodiment 3, the present invention is directed to the composition of embodiment 2, further comprising at least one surfactant compound.

In embodiment 4, the present invention is directed to the composition of embodiment 3, where the at least one surfactant compound represents less than 20% of the total weight of the combination of the at least one aqueous-insoluble compound plus the at least one bile acid compound plus the at least one surfactant compound in the nanoparticles.

In embodiment 5, the present invention is directed to the composition of embodiment 3, where the at least one surfactant compound is a non-cationic surfactant compound.

In embodiment 6, the present invention is directed to the composition of embodiment 3, where the combined weights of the at least one aqueous-insoluble compound plus the at least one bile acid compound plus the at least one surfactant represent at least 90% of the weight of the nanoparticles in the composition.

In embodiment 7, the present invention is directed to the composition of embodiment 1, where the at least one aqueous-insoluble compound is a pharmaceutically useful compound selected from the group consisting of a therapeutic and/or diagnostic compound and a contrast agent.

In embodiment 8, the present invention is directed to the composition of embodiment 7, where the at least one pharmaceutically useful compound is a therapeutic and/or diagnostic compound.

In embodiment 9, the present invention is directed to the composition of embodiment 8, where the therapeutic and/or diagnostic compound is selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators and xanthines.

In embodiment 10, the present invention is directed to the composition of embodiment 1, where the composition has a polydispersity index (“PDI”) selected from the group consisting of a PDI of less than about 1.0, 0.8, 0.6, and 0.4.

In embodiment 11, the present invention is directed to the composition of embodiment 10, where the composition has a PDI of less than about 0.4.

In embodiment 12, the present invention is directed to the composition of embodiment 1, where the nanoparticles have a mean diameter selected from the group consisting of a mean diameter of less than about 10 μm, 1 μm, 0.5 μm, and 0.2 μm.

In embodiment 13, the present invention is directed to the composition of embodiment 12, where the nanoparticles have a mean diameter of less than about 0.2 μm.

In embodiment 14, the present invention is directed to the composition of embodiment 1, where the composition has a PDI of less than about 0.4, and the nanoparticles have a mean diameter of less than about 1 μm.

In embodiment 15, the present invention is directed to the composition of embodiment 1, where the nanoparticles comprise a aqueous-insoluble compound in a crystalline form, a non-crystalline form, or a combination of crystalline and non-crystalline forms.

In embodiment 16, the present invention is directed to the composition of embodiment 15, where the nanoparticles comprise an aqueous-insoluble compound in substantially non-crystalline form.

In embodiment 17, the present invention is directed to the composition of embodiment 1, where the at least one bile acid compound is a steroid acid, or salt thereof, including cholic acid, taurocholic acid, glycocholic acid, lithocholic acid, chenodeoxycholic acid, deoxycholic acid, glycodeoxycholic acid, taurodeoxycholic acid, derivatives thereof, and mixtures thereof.

In embodiment 18, the present invention is directed to a pharmaceutical formulation comprising the composition of any one of the preceding embodiments.

In embodiment 19, the present invention is directed to the formulation of embodiment 18, where the nanoparticles are dispersed in a tablet, capsule, ointment, cream, film or

lyophilized powder/formulation In embodiment 20, the present invention is directed to a method of treating a mammal comprising administering to a mammal an effective amount of the formulation of embodiment 19.

In embodiment 21, the present invention is directed to the use of the formulation of embodiment 19 for the treatment of a disease in a mammal.

In embodiment 22, the present invention is directed to the use of the formulation of embodiment 19 to make a medicament to treat a disease susceptible to the drug.

In embodiment 23, the present invention is directed to a method of forming nanoparticles, comprising combining a solution of at least one aqueous-insoluble compound in a first solvent with a miscible precipitation solution comprising at least one bile acid compound so as to form nanoparticles comprising the at least one aqueous-insoluble compound.

In embodiment 24, the present invention is directed to the method of embodiment 23, where the miscible precipitation solution further comprises at least one surfactant compound.

In embodiment 25, the present invention is directed to the composition of embodiment 24, where the at least one surfactant compound is a non-cationic surfactant.

### DETAILED DESCRIPTION OF THE INVENTION

The present invention is directed to methods of preparing nanoparticles of aqueous-insoluble compounds, particularly aqueous-insoluble bioactive (drug) compounds, and to compositions and medicaments obtained by these methods. These methods, compositions, and other inventive aspects of the present invention are based particularly on the use of bile acid compound(s) to prepare nanoparticles of aqueous-insoluble compounds, since the Applicants have discovered that these bile acid compound(s) unexpectedly decrease the average size of such nanoparticles that can be obtained by currently utilized methodologies, including particularly non-emulsion methods such as solvent/anti-solvent processes such as, e.g., those of Violante et al., and also confer additional unexpected and advantageous properties, e.g., a narrower distribution of nanoparticle sizes.

Thus with regard to average nanoparticle size, as discussed elsewhere herein and as shown in the non-limiting Examples of the present invention, the Applicants have discovered to that including bile acid compound(s) in methods used for preparing compositions of nanoparticles of aqueous-insoluble compounds, e.g., solvent/anti-solvent processes such as those discussed in U.S. Pat. Nos. 4,826,689 and 4,997,454 to Violante et al., unexpectedly produce nanoparticles that are molecular aggregates, for example, on average as much as 5×-10× smaller than are obtained using the previous methods without bile acid compound(s). For example, Example 4 shows that the conventional solvent/anti-solvent preparation of nanoparticles of the drug diclofenac with the addition of the surfactant Lipoid S-45 produces nanoparticles of average size of 2564 nm, while addition of Lipoid S-45 in combination with the bile acid compound sodium deoxycholate (“DOC”) unexpectedly results in 5× smaller particles of average size of 453 nm, while Example 2 shows that the addition of DOC with the surfactant sodium lauryl sulfate (SLS) in a diclofenac preparation unexpectedly results in a greater than 10× reduction of nanoparticle size over SLS alone, i.e., an average size of 390 nm with SLS +DOC versus an average size of 5542 nm with SLS only.

Bile acid compound(s) are also used in the present invention because, in addition to the above discovery regarding the effects of these compounds on average nanoparticle size, the Applicants have also unexpectedly found that the use of these compounds to produce nanoparticles results in a particularly narrow size distribution of the resulting nanoparticles. Thus for example the size range or “polydispersity index” (“PDI”; see elsewhere for definition) of the diclofenac nanoparticles obtained in Example 4 with Lipoid S-45 is 0.525, i.e., the nanoparticles have a wide size range (are “polydispersed”). By contrast, the PDI for diclofenac nanoparticles obtained using the combination of Lipoid S-45 +DOC is 0.197, i.e., significantly closer to having a uniform size (i.e., being “monodisperse”), a situation that occurs when the PDI is less than 0.1. Similarly improved particle size distributions are seen in the other Examples, with Example 3 showing an instance of diclofenac nanoparticles obtained with polyvinyl alcohol +DOC that have a PDI so low (0.018) as to indicate uniformity of size (monodispersity).

These results presented above with regard to size range are particularly significant because tightly-controlled size ranges correlate with better pharmaceutical properties, e.g., with tightly-controlled dissolution rates (since more-consistently-sized particles will tend to dissolve more similarly than will particles of widely varying size), better shelf stability (as the more uniformly sized particles are less susceptible to Ostwald ripening), and better efficacy/tissue targeting (because of the more uniform bio-distribution). See, e.g., U.S. Pat. No. 4,826,689. Thus the unexpected ability to produce more consistently sized nanoparticles by the addition of bile acid compound(s) is highly significant in terms of the preparation of better drug formulations.

With regard to the unexpected results discussed above for nanoparticle size and polydispersity obtained using the bile acid compounds (s) of the present invention, Applicants note that the primary biological function of bile acid(s) is in emulsifying fat into micelles, thus aiding in fat processing. In light of this known biological role, various previous workers have produced emulsions of drug compounds with phospholipids or emulsifiers such as ethylcellulose and with particular bile acid compounds, in order to produce drug-containing nanoparticles. See, e.g., WO2008/125940, for a description of the use of drug+phospholipid+bile acid compound(s) and, e.g., WO2008/135828, for a description of the use of drug+the emulsifier ethylcellulose+bile acid compound(s). The contents of both of these references are herein incorporated in their entireties by reference. However, these references use a bile acid compound in the emulsification process to stabilize the final nanoparticles obtained, rather than to obtain exceptionally small nanoparticles or tight particle size distributions from the outset. Thus for example WO2008/135828 provides data that show that, for the emulsification conditions used, the end products are stabilized by bile acid compound (see, e.g., Control 1, page 31, Control 2, page 37, and Table 9 on the same page), and duly notes this anti-agglomerative role for bile acid compound (see page 37, lines 24-27). Thus this reference specifically states that, with regard to the role of bile compounds, “the bile salt helps promote stability of nanoparticle suspensions, reducing, slowing, or preventing agglomeration of the nanoparticles” (see page 2, lines 31-32).

By contrast and as already discussed, in the present invention, the Applicants have discovered a critical—and unexpected—role for bile acid compound(s) in the formation itself of small particles of tight size distribution, a recognition that is different from the previously observed results on the anti-agglomeration of already-formed nanoparticles. Applicants also note that there are significant differences between the nanoparticle formation methods used in the previous references (emulsification, not the exemplary solvent/anti-solvent process of the present invention), as well as fundamental differences in the nanoparticles produced (e.g., high bile salt amount as compared to the present invention and low drug loading as compared to the present invention). As discussed below, the present invention exploits this observation of these unexpected properties of bile acid compound(s) to provide advantageous methods of forming nanoparticles, as well as to nanoparticles with very different compositions from those described in, e.g., the cited references.

Nanoparticles. With regard to terminology, the term “nanoparticle” or “nanoparticles” refers to a particle or particles produced by the methods of the present invention that either may be used by themselves or, preferably, in “formulations,” i.e., compositions comprising the nanoparticles and other compound(s) that are optimized for delivery of the nanoparticles, e.g., drug delivery. The nanoparticles of the invention have a characteristic dimension (generically, “size”), such as average diameter, of less than 10 μm, e.g., less than 10, 5, 2, or 1 μm. In a preferred embodiment, the nanoparticles of the invention are in a range of 0.01 μm (10 nm) to about 5 μm (5000 nm) of average diameter (synonymously, “average size”). In another preferred embodiment, the nanoparticles of the invention have a characteristic average diameter of less than about 1 μm, i.e., are in the nm size range.

For embodiments of the present invention specifying an average diameter of nanoparticles, this average diameter may be determined as the “average effective particle diameter,” which may be measured by, e.g., dynamic light scattering methods (e.g., photon correlation spectroscopy, laser diffraction, low-angle laser light scattering (LALLS), medium-angle laser light scattering (MALLS), rheology, or microscopy (light or electron).

Crystalline/Non-Crystalline/Amorphous. As already discussed, the nanoparticles of the invention comprise at least one aqueous-insoluble compound (but can also include a mixture of more than one of such compounds) and, preferably, a bile acid compound or bile acid compounds. In some embodiments a surfactant or surfactants are also included; in other embodiments, other compounds may also be present. In some embodiments, the aqueous-insoluble compound(s) component of the nanoparticles is/are substantially crystalline; in other embodiments, the compound or compounds is/are substantially non-crystalline.

The term “substantially crystalline” refers to a situation where a high percentage of the aqueous-insoluble compound or compounds present in the nanoparticles exhibit long-range order in three dimensions e.g., a distance of more than a few molecules. “Substantially non-crystalline” or, synonymously, “substantially amorphous” refers to a situation where a high percentage of the compound or compounds lack long-range three-dimensional order, and includes not only material which has essentially no order, but also material which may have some small degree of order, but the order is in less than three dimensions and/or is only over short distances, e.g., a distance of a few molecules. The present invention is particularly directed to substantially non-crystalline/substantially amorphous drug compound or compounds situations, since the non-crystalline/amorphous form of a low-solubility drug provides a greater aqueous concentration of drug relative to the crystalline form of the drug in an aqueous use environment.

Thus the present invention is particularly directed to aqueous-insoluble compounds(s) in nanoparticles, where the compound(s) are substantially non-crystalline, i.e., a high percentage of the aqueous-insoluble compound or compounds in the nanoparticles is/are in non-crystalline form, e.g., at least about 70%, 71%, 72%, . . . 97%, 98%, 99%, etc. of the compound or compounds in the nanoparticles is/are in non-crystalline form. The degree of non-crystallinity is preferably evaluated as a percentage of the compound or compounds that are non-crystalline in the collection of nanoparticles as a whole (i.e., as a function of the bulk of nanoparticles), i.e., when at least about 70%, 71%, 72%, . . . 97%, 98%, 99%, etc. by weight of the total weight of aqueous-insoluble compound or compounds in a preparation of nanoparticles are in non-crystalline form. However, in addition to being expressed as a wt %, the percentage may in some situations be measured based on individual or only small numbers of nanoparticles (i.e., non-bulk percentages). Alternatively, the degree of non-crystallinity may be expressed as a limit on the maximum amount of crystalline compound organization in a sample, e.g., no more than about 20, 19, 18, . . . 3, 2, 1%, etc., of crystallinity. Amounts of crystalline compound may be measured by Polarized light microscopy, Powder X-Ray Diffraction (PXRD), Differential Scanning calorimetry (DSC), solid-state nuclear magnetic resonance (NMR), or by any other appropriate measurement.

Polydispersity Index. The term “polydispersity index” is defined as a measure of the distribution broadness of a sample, and is typically defined as the relative variance in the correlation decay rate distribution, as is known by one skilled in the art. See B J. Fisken, “Revisiting the method of cumulants for the analysis of dynamic light-scattering data,” Applied Optics, 40(24), 4087-4091 (2001) for a discussion of cumulant diameter and polydispersity. Preferably, the polydispersity of the nanoparticles is less than 0.8, preferably less than 0.5, and more preferably less than 0.3 and most preferably less than 0.2.

Aqueous-Insoluble Compound(s). The present invention is directed to the production of nanoparticles of aqueous-insoluble compounds. As used herein, the term “aqueous-insoluble compound(s)” (synonymously, “water insoluble compound(s)”) refers to organic compounds, including organometallics, which are solids under conditions of standard temperature and pressure (23° C., 1 Atm), and preferably have limited aqueous solubility. The term “limited aqueous solubility” refers to compounds that have a water-solubility of less than about one part in one thousand (g solute per ml solvent) and preferably less than about one part in ten thousand. Definitions of solubilities that may be referred to in the present invention include (in g solute per ml solvent) “very soluble” (less than 1 part solvent needed to dissolve 1 part solute); “freely soluble” (from 1 to 10 parts solvent needed to dissolve 1 part solute); “soluble” (from 10 to 30 parts solvent needed to dissolve 1 part solute); “sparingly soluble” (from 30 to 100 parts solvent needed to dissolve 1 part solute); “slightly soluble” (from 100 to 1000 parts solvent needed to dissolve 1 part solute); “very slightly soluble” (from 1000 to 10,000 parts solvent needed to dissolve 1 part solute); and, “practically insoluble” (more than 10,000 parts solvent needed to dissolve 1 part solute). Thus the limited solubility of aqueous-insoluble compounds that is referred to herein is particularly directed to slightly soluble compounds and those with even less solubility (e.g., very slightly soluble and practically insoluble compounds).

Preferred aqueous-insoluble compounds are those having a solubility in water less than 5 mg/ml at a physiological pH of 6.5 to 7.4, preferably less than 1 mg/ml and more preferably less than 0.1 mg/ml. (10 mg/ml). These aqueous-insoluble compounds are preferably “pharmaceutically useful” compounds, e.g., drug or imaging compounds, but explicitly include non-pharmaceutical compounds, e.g., inks, pigments and paints. Pharmaceutically useful, aqueous-insoluble organic compounds include any organic chemical entities whose solubility decreases from one solvent to another. The organic compounds may be biologically useful compounds, imaging agents, and pharmaceutically useful compounds. Alternatively, the organic compound might be from the group used as adjuvants or excipients in pharmaceutical preparations and cosmetics, such as, but not limited to, preservatives, e.g., propylparaben. Pharmaceutical compounds include drugs for human and veterinary medicine.

Examples of aqueous-insoluble drugs include, but are not limited to, analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, alpha & beta-adrenoceptor blocking agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators, bronchodilators(xanthines), xanthenes, neutraceuticals and cosmeceuticals. Applicants note that each named drug provided herein should be understood to include the nonionized form of the drug or pharmaceutically acceptable forms of the drug. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

Other non-limiting examples of aqueous-insoluble drugs include immunosuppressive agents such as cyclosporines including cyclosporine A, tacrolimus, and mycophenolate mofetil; immunoactive agents, antiviral and antifungal agents, antineoplastic agents, analgesic and anti-inflammatory agents, antibiotics, anti-epileptics, anesthetics, hypnotics, sedatives, antipsychotic agents, neuroleptic agents, antidepressants, anxiolytics, anticonvulsant agents, antagonists, neuron blocking agents, anticholinergic and cholinomimetic agents, antimuscarinic and muscarinic agents, antiadrenergic and antiarrhythmics, antihypertensive agents, antineoplastic agents, hormones, antihyperlipidemics; antimicrobials, e.g., antibacterials such as sulfadiazine, antifungals such as itraconazole; non-steroidal anti-inflammatory drugs, e.g., indomethacin; antihypercholesteremic agents, e.g., probucol; and steroidal compounds, e.g., dexamethasone; and nutrients. A detailed description of these and other suitable drugs may be found in Remington, The Science and Practice of Pharmacy 20th edition, 2000, Lippincott, Williams & Wilkens. Baltimore.

Applicants note that “a” and “an” as used herein are explicitly not intended as specifying a single instance, and instead specify at least one instance, i.e., one or more instances. Thus for example “a compound” refers to at least one compound, including one, two, three, etc. compounds, “an aqueous-insoluble compound” refers to at least one such compound, including one, two, three, etc. compounds, etc. Applicants also note that, when “a” or “an” is used to describe a component of a composition representing some % fraction of that composition, the % fraction is applied to that component either as a single instance or as multiple instances. Thus for example, when “an aqueous-insoluble compound” is said to represent some fraction of the weight of the nanoparticles in a composition, that fraction is either the fraction of the weight provided by a single aqueous-insoluble compound or, when there are multiple such compounds, for the weight of all of those compounds combined. Effective Amount. The term “effective amount” relates to the amount of a drug that persons of skill in the relevant medical, dental, or veterinary art would recognize as effective to treat a disease. As such, an effective amount relates to the specific combination of drug and disease or medical condition in question. An effective amount of a drug can be determined either by previous data on the dosage/dosing of a drug required in combination with, e.g., experimental assays as to rate of drug delivery, bioavailability, etc.

In one embodiment, the compositions of the present invention are capable of improving the concentration of dissolved drug in a use environment relative to a control composition of a suitably formulated drug as prepared by standard methods, i.e., not by the methods of the present invention. Various methods may be used to determine such concentration enhancement in vitro, e.g., the amount of “free” drug, or solvated drug may be measured. By “free” drug is meant drug which is in the form of dissolved drug but which is not in the nanoparticles or any solid particles larger than 500 nm. A composition of the invention provides concentration enhancement if, when administered to an aqueous use environment, it provides a free drug concentration that is at least 1.25-fold the free drug concentration of the control composition. Preferably, the free drug concentration provided by the compositions of the invention are at least about 1.5-fold, more preferably at least about 2-fold, and most preferably at least about 3-fold that provided by the control composition.

Alternatively, the compositions of the present invention, when administered to a human or other animal, provide an AUC in drug concentration in the blood plasma or serum (or relative bioavailability) that is at least 1.25-fold that observed in comparison to the control composition. Preferably, the blood AUC is at least about 2-fold, more preferably at least about 3-fold, even more preferably at least about 4-fold, still more preferably at least about 6-fold, yet more preferably at least about 10-fold, and most preferably at least about 20-fold that of the control composition. The determination of AUCs is a well-known procedure and is described, for example, in Welling, “Pharmacokinetics Processes and Mathematics,” ACS Monograph 185 (1986).

Alternatively, the compositions of the present invention, when administered to a human or other animal, provide a maximum drug concentration in the blood plasma or serum (Cmax) that is at least 1.25-fold that observed in comparison to the control composition. Preferably, the Cmax is at least about 2-fold, more preferably at least about 3-fold, even more preferably at least about 4-fold, still more preferably at least about 6-fold, yet more preferably at least about 10-fold, and most preferably at least about 20-fold that of the control composition. Thus, compositions that meet the in vitro or in vivo performance criteria, or both, are considered to be within the scope of the invention.

Exemplary Solvent/Anti-Solvent Method of Nanoparticle Preparation. The present invention relates to the preparation of aqueous-insoluble compounds as uniform particles, and is particularly advantageous in its increased ability to control the average size and uniformity of the particles.

The present invention contemplates a variety of methods for nanoparticle preparation, particularly non-emulsion methods, with solvent/anti-solvent methods being particularly preferred, e.g., the miscible solvent/anti-solvent methods discussed in U.S. Pat. Nos. 4,826,689 and 4,997,454 to Violante et al., or as known to one or ordinary skill in the art of such solvent/anti-solvent methods. Thus in a preferred method of the present invention, an aqueous-insoluble compound is dissolved in a first solvent. Particles are formed by a controlled solvent displacement process, in which an aqueous phase second solvent (synonymously, “anti-solvent”) displaces the first solvent, thus developing molecular solid aggregate based suspension.

In such a process, the first step is typically to prepare a solution of the compound of interest in a first solvent, e.g., an organic solvent or solvent mixture suitable for that compound. This can occur as the compound is synthesized as a dissolved solid, or by dissolving the compound in the solvent of choice. The compound is typically added at from about 0.1% (w/v) to about 50% (w/v) depending on the solubility of the organic compound in the first solvent. In some instances heating from about 30° C. to about 100° C. may be necessary to ensure total dissolution of the compound in the first solvent.

In an optional but preferred step, the first solvent may be diluted with a dilution solution, a non-solvent that does not cause the compound to precipitate. The dilution solution causes greater dispersion of the dissolved molecules of the compound in the liquid phase. Greater dilution of the solution with non-solvent produces larger particles/ molecular solid aggregates, and less dilution of the solution with non-solvent produces smaller particles/molecular solid aggregates. Such tuning of particle size is particularly desirable in order to obtain a size that affords the desired delivery rate, amount, etc., of the aqueous-insoluble compound(s), e.g., the desired delivery rate of an aqueous-insoluble drug compound or compounds.

The “first solvent” may be a solvent or mixture of solvents in which the compound or compounds is/are relatively soluble, and which is miscible with the second solvent. Examples of such first solvents include, but are not limited to, N-methyl-2-pyrrolidinone (also called N-methyl-2-pyrrolidone), 2-pyrrolidone, dimethyl sulfoxide, dimethylacetamide, lactic acid, methanol, ethanol, isopropanol, 3-pentanol, n-propanol, glycerol, butylene glycol (butanediol), ethylene glycol, propylene glycol, mono- and diacylated monoglycerides (such as glyceryl caprylate), dimethyl isosorbide, acetone, dimethylformamide, 1,4-dioxane, polyethylene glycol (for example, PEG-4, PEG-8, PEG-9, PEG-12, PEG-14, PEG-16, PEG-120, PEG-75, PEG-150, polyethylene glycol esters (examples such as PEG-4 dilaurate, PEG-20 dilaurate, PEG-6 isostearate, PEG-8 palmitostearate, PEG-150 palmitostearate), polyethylene glycol sorbitans (such as PEG-20 sorbitan isostearate), polyethylene glycol monoalkyl ethers (examples such as PEG-3 dimethyl ether, PEG-4 dimethyl ether), polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate, etc.

Surfactants. The next step in standard solvent/anti-solvent methods of forming nanoparticles is to precipitate the aqueous-insoluble compound or mixture of compounds from the solution in a desired particle size by mixing with a second solvent comprising an aqueous solution of a surfactant or surfactants, in sufficient quantity that this second solvent effects substantially complete precipitation of the compound or compounds. In such standard solvent/anti-solvent methods, anionic, cationic, and nonionic surfactants are considered equally suitable components of the second solvent to cause precipitation. See, e.g., U.S. Pat. No. 6,607,784 and U.S. Patent Publication No. 2006/0222711, the contents of which are herein incorporated in their entireties by reference.

In the present invention, however, where bile acid compounds are used to obtain the unexpected results of nanoparticle size and tightness of size range, either alone or preferably in combination with one or more surfactants, the use of cationic surfactants is disfavored, because they tend to form ionic complexes with such bile acid compound(s). Therefore, although some embodiments of the present invention do explicitly include the use of such cationic surfactants, these cationic surfactants are preferably excluded from the “surfactants” normally contemplated. Thus preferred embodiments of the present invention are directed to the use of either bile acid compound or compounds alone, or preferably such a compound or compounds in combination with one or more non-cationic surfactants, i.e., with anionic or nonionic surfactants.

Thus in light of the above, the term “surfactant(s)” is defined to refer to surface property modifying agents including tensides, detergents, and surfactants, where these agents can include cationic surfactants, while “non-cationic surfactant(s)” refers to all such surfactant compounds that are explicitly non-cationic.

Non-limiting examples of suitable “non-cationic surfactants” include (a) natural surfactants such as casein, gelatin, tragacanth, waxes, enteric resins, paraffin, acacia, gelatin, cholesterol esters and triglycerides, albumin, heparin, hirudin or other appropriate proteins; (b) nonionic surfactants such as polyoxyethylene fatty alcohol ethers (Macrogol and Brij), sorbitan fatty acid esters (Polysorbates), polyoxyethylene fatty acid esters (Myij), sorbitan esters (Span), glycerol monostearate, polyethylene glycols, cetyl alcohol, cetostearyl alcohol, stearyl alcohol, aryl alkyl polyether alcohols, polyoxyethylene-polyoxypropylene copolymers (poloxamers), polaxamines, methylcellulose, hydroxycellulose, hydroxy propylcellulose, hydroxy propylmethylcellulose, noncrystalline cellulose, polysaccharides including starch and starch derivatives such as hydroxyethylstarch (HES), polyvinyl alcohol, polyvinylpyrrolidone, and synthetic phospholipids; (c) anionic surfactants such as potassium laurate, triethanolamine stearate, sodium lauryl sulfate, sodium dodecylsulfate, alkyl polyoxyethylene sulfates, sodium alginate, dioctyl sodium sulfosuccinate, negatively charged or desalted, hydrogenated or partially hydrogenated or natural semisynthetic or synthetic phospholipids (phosphatidyl glycerol, phosphatidyl inosite, phosphatidylserine, phosphatidylcholine, phosphatidylethanolamine, phosphatidylinositol, phosphatidic acid, lysophospholipids, egg or soybean phospholipid or a combination thereof, phosphatidic acid and their salts), and negatively charged glyceryl esters, sodium carboxymethylcellulose, and calcium carboxymethylcellulose; (d) colloidal clays such as bentonite and veegum or a combination thereof. A detailed description of these surfactants may be found in the aforementioned Remington's Pharmaceutical Sciences, and Theory and Practice of Industrial Pharmacy, Lachman et al, 1986.

Bile-Acid Compound(s). The present invention is based on the unexpected discovery that bile acid compound(s) advantageously affect nanoparticle size and size distributions. The term “bile acid compound(s)” refers to compounds including, but not limited to, a steroid acid, or salt thereof, including cholic acid, taurocholic acid, glycocholic acid, lithocholic acid, chenodeoxycholic acid, deoxycholic acid, glycodeoxycholic acid, derivatives thereof, and mixtures thereof. The term is intended to encompass any such compound recognized by a person of skill in the art as a bile-acid or a cholate derivative. Thus for example, the non-limiting example of WO2008/125940 (page 13) states that “Bile salts are the acid addition salts of bile acids. The bile acids are divided into two groups: primary (derived from cholesterol) and secondary (derived from primary bile acids). The bile salts are conjugated through peptide linkages to glycine or taurine. The primary bile salts are taurine or glycine conjugates of cholic acid or chenic acid; the secondary bile salts are taurine and glycine conjugates of deoxycholic and lithocholic acids. See Remington the Science and Practice of Pharmacy (2^{0th} edition, 2000, at page 1228). The term ‘bile salt’ includes mixtures of bile salts. Exemplary bile salts include the salts of dihydroxy cholic acids, such as deoxycholic acid, glycodeoxycholic acid, taurodeoxycholic acid, chenodeoxycholic acid, glycochenodeoxycholic acid, and taurochenodeoxycholic acid, and trihydroxy cholic acids, such as cholic acid, glycocholic acid, and taurocholic acid. The acid addition salts include sodium, and potassium.” See, e.g., U.S. Pat. No. 5,057,509, for various non-limiting examples.

Although not bound by any particular theory, Applicants believe that, in light of the observations described herein, it is reasonable to think that surface modifiers in general adsorb to the surfaces of drug particles, and that the presence of bile acid compound(s) may produce molecular aggregates manifesting as small particles possibly by facilitating the formation of micelles early in the particle growth phase and/or modifying the zeta potential of surfaces with more charge repulsion stabilization sooner in the particle growth phase while surface modifiers in general adsorb to the surfaces of drug particles stabilizing particles later in the growth phase. Thus on the basis of the unexpected results presented here, and on the basis of the conclusions drawn after the availability of these results—and based on these results—it may be possible to explain the observed effect of bile acid compounds on, e.g., initial nanoparticle sizes produced, and possibly to select other compounds having similarly advantageous properties as the bile acid compounds described herein. This is in addition to previous observations by others regarding bile acid compounds and their anti-agglomerative effects (see previous discussion). Although not bound by any particular theory regarding this anti-agglomerative effect, it is possible that this effect is obtained via the promotion by bile acid compounds of the formation of micelles, thereby inhibiting further particle growth.

Precipitation To Form Nanoparticles. As already discussed, in the present invention one or more aqueous-insoluble compounds in a suitable first solvent are combined with a second solvent comprising one or more bile acid compounds and zero or more surfactants in order to form the nanoparticles of the present invention.

With regard to this precipitation step involving the addition of the second solvent comprising bile acid compound(s)/surfactant(s) (collectively, the “precipitation solution”), the solvent used may be water, or a solvent other than water, so long as it is a miscible non-solvent for the dissolved organic compound and is a solvent for the bile acid compound(s) present. Preferably, precipitation is performed under conditions of controlled pH. Preferably the pH is within a range of from about 3 to about 11, and is controlled with a pH-adjusting agent such as, but not limited to, sodium hydroxide or hydrochloric acid. Preferably, the precipitation solution is buffered to a desired pH value using a buffer including, but not limited to tris, citrate, acetate, lactate, meglumine, or similar pH-buffers. The addition rate for mixing of the solutions to cause precipitation is dependent on the batch size, and the solvent displacement kinetics for the organic compound. Typically, for a small-scale laboratory process (preparation of 1 liter), the addition rate is from about 0.05 ml per minute to about 10 ml per minute. During the addition, the solutions should be under constant agitation. Although mixing can occur under a variety of conditions, preferably mixing occurs at a temperature between about −1° ° C. and about 10° ° C. In preferred aspects, the mixing occurs at a rate of from about 0.01 ml per min. to about 1000 ml per min. per 50 ml unit volume of solution.

The choice of bile acid compound(s) and optional surfactant(s) to be included in the precipitation solution will depend upon the aqueous-insoluble compound or mixture of compounds selected, and will be determined based on the desired outcome of nanoparticle size, size distribution, etc. For example, any empirical test used to assay the size and size distribution of the nanoparticles produced may be used, e.g., photon correlation spectroscopy and/or other tests disclosed in the Examples and elsewhere herein, in WO2008/135828, etc.

Nanoparticle Composition. The nanoparticles produced by the methods of the present invention contain at least one aqueous-insoluble compound, preferably a bile acid compound or compounds, and, optionally, one or more surfactants. As described above, one characteristic of these nanoparticles is the extent of crystallinity/non-crystallinity of the aqueous-insoluble compound or compounds comprising the bulk nanoparticles produced. However, there are a number of other important parameters that are used to characterize these nanoparticles, particularly the wt % content of the bulk nanoparticles of aqueous-insoluble compound(s), bile acid compound(s), and surfactant(s).

The nanoparticles of the present invention are preferably characterized by a high wt % of aqueous-insoluble compound or compounds, e.g., at least about 76 wt %, 77 wt %, 78 wt %, 79 wt %, 98 wt %, 99 wt %, etc. of the weight of the solid components of the nanoparticles are preferably aqueous-insoluble compound or compounds (e.g., at least about 76 gram per 100 gram of solid components are aqueous-insoluble compound(s), at least about 77 gram per 100 gram are aqueous-insoluble compound(s), etc.).

Thus the nanoparticles will typically contain at least about 80 wt %, 81 wt %, 82 wt %,

. . . , 97 wt %, 98, 99 wt %, etc. solid components, e.g. In a preferred embodiment, at least 80 wt %, 81 wt % 82 wt %, . . . , 97 wt %, 98, 99 wt %, etc. of the weight of nanoparticles is provided by a combination of the solid components: aqueous-insoluble compound(s), bile acid compound(s), and, optionally, surfactant(s). In this embodiment, at least about 76 wt %, 77 wt %, 78 wt %, 79 wt %, . . . 97 wt %, 98 wt %, etc. of the weight of these solid components in the nanoparticle is preferably aqueous-insoluble compound(s). More preferably, the wt % of the aqueous-insoluble compound or compounds is greater than at least about 95 wt %. When the nanoparticles of the invention comprise more than one aqueous-insoluble compound, the wt % referred to is for the combination of compounds.

When the aqueous-insoluble compound(s) are drug compound(s), the amount of drug compound or compounds in the bulk of the nanoparticles is referred to as “drug loading.” Contemplated wt % values for drug loading are as provided for aqueous-insoluble compounds in general as discussed above, e.g., at least about 76 wt %, 77 wt %, 78 wt %, 79 wt %, . . . 97 wt %, 98 wt %, etc. of the drug compound or compounds, and more preferably greater than at least about 95 wt % of the drug compound(s). Applicants note that the drug loading obtained in the present invention is relatively high versus earlier work; W02008/135828, for example, recites a maximum of about 75 wt % drug (see, e.g., page 6, linel 9), with an amount of only up to about 60 wt % stated as being most preferable (page 6, line 22). Applicants note that, as for this reference, the wt % values provided herein refer to weight of compound(s) relative to the total mass of the in the nanoparticles.

The nanoparticles of the present invention also preferably contain at least one bile acid compound. In one embodiment of the present invention, the added bile acid compound(s) is/are retained in the nanoparticles produced; in other embodiments this compound or compounds may be at least partially removed from the nanoparticles subsequent to nanoparticle production by, e.g., tangential flow filtration. With regard to the amount of bile acid compound(s) present in the bulk weight of the nanoparticles, typical amounts range from 20 wt %, 19 wt %, 18 wt %, 1 wt % (counting by 1 wt % decrements), etc., more preferably less than about 10 wt %, and still more preferably less than about 5 wt %, 4.9 wt %, 4.8 wt %, . . . (counting by 0.1 wt % decrements), etc., of the total mass of the solids in the nanoparticles.

Nanoparticles of the present invention may also optionally contain one or more surfactants. In this regard, surfactant(s) are typically present in the range of about 0.01 to 24 wt %, preferably 0.1 to 20 wt %, more preferably 0.1 to 10 wt %, and still more preferably 0.1 to 1 wt %. These, wt % values are determined as weight of surfactant as a percentage of the combined weight of solids in the nanoparticles, i.e., in the same manner as given above for aqueous-insoluble compound(s) or bile acid compound(s).

Formulations. The compounds of the invention may be administered alone, or preferably in compositions (synonymously, “formulations”) using any known dosage form. The compositions comprising nanoparticles may be formulated for administration via oral, topical, subdermal, intranasal, buccal, intrathecal, ocular, intraaural, subcutaneous spaces, intraarticular, vaginal tract, arterial and venous blood vessels, pulmonary tract or intramuscular tissue of an animal, such as a mammal and particularly a human. Oral dosage forms include: powders or granules; tablets; chewable tablets; capsules; unit dose packets, sometimes referred to in the art as “sachets” or “oral powders for constitution” (OPC); syrups; and suspensions. Parenteral dosage forms include reconstitutable powders or suspensions. Topical dosage forms include creams, pastes, suspensions, powders, foams and gels. Ocular dosage forms include suspensions, powders, gels, creams, pastes, solid inserts and implants.

Formulations prepared by this invention may be dried into powders by lyophilization, by fluid or spray drying, or other suitable means known to those skilled in the art. Powders may be suspended in solution, filled into capsules, or converted to granular or tablet form with the addition of binders and other excipients known in the art of tablet making.

Stabilization of Formulations/Anti-Aggregants. Various additional compounds may be included in the precipitation and isolation of the nanoparticles of the present invention in order to stabilize these nanoparticles in formulations, to control aggregation, etc. Non-limiting examples of suitable to these purposes include the following surfactants, taken singly or in combination: polaxomers, such as Pluronic™ F68, F108 and F127, which are block copolymers of ethylene oxide and propylene oxide available from BASF, and poloxamines, such as Tetronic™ (T908), which is a tetrafunctional block copolymer derived from sequential addition of ethylene oxide and propylene oxide to ethylene-diamine available from BASF, Triton™ X-200, which is an alkyl aryl polyether sulfonate, available from Rohm and Haas. Among polyoxyethylene fatty acid esters is included those having short alkyl chains. One example of such a surfactant is SOLUTOL™ HS 15, polyethylene-660-hydroxystearate, manufactured by BASF Aktiengesellschaft. Tween 20, 40, 60 and 80, which are polyoxyethylene sorbitan fatty acid esters, available from ICI Specialty Chemicals, polyoxyethylene stearate (Myri 52) available from ICI Specialty Chemicals, Carbowax™ 3550 and 934, which are polyethylene glycols available from Union Carbide, hydroxy propylmethylcellulose, dimyristoyl phosphatidylglycerol sodium salt, sodium dodecylsulfate. In some cases it is preferred that at least two surfactants are used. In a preferred aspect of the invention, where free-flowing formulations are desired, the surfactant(s) will itself is preferably a powder.

Aspects of the invention are further illustrated by the following experiments, which are not meant to limit the scope of the invention. In all of these Examples, particle size and PDI were determined using photon correlation spectroscopy, and crystallinity was determined by polarized light microscopy.

### EXAMPLE 1

A control precipitation was performed by rapidly mixing 0.25 ml of 33 mg/ml metolazone in THF with 10 ml aqueous 0.10% polymer at room temperature. The resultant suspension consisted of 3 gm crystals of metolazone. Controlled precipitations were performed by rapidly mixing 0.25 ml of 33 mg/ml metolazone in THF with 10 ml aqueous 0.05% polymer and 0.05% bile acid compounds. The resultant suspensions of amorphous metolazone particles are as follows: sodium deoxycholate—233 nm (PDI=0.112); sodium taurodeoxycholate—221 nm (PDI=0.110); sodium cholate—378 nm (PDI=0.175) and sodium taurocholate—266 nm (PDI=0.131). Bile acid compounds yield nanoparticle suspensions that are less than 400 nm with PDI's below 0.20.

### EXAMPLE 2

A control precipitation was performed by rapidly mixing 0.25 ml of 30 mg/ml diclofenac in ethanol with 10 ml aqueous 0.1% (w/v) sodium lauryl sulfate at room temperature (RT). The resultant suspension consisted of 5542 nanometer crystals (average dimension), as measured by photon correlation spectroscopy, diclofenac particles having a 2.013 polydispersity index (PDI). Experimental precipitations were performed by mixing, under the same conditions, 0.25 ml of 30 mg/ml diclofenac with 10 ml aqueous 0.05% sodium lauryl sulfate and 0.05% sodium deoxycholate resulting in a suspension of 390 nm amorphous diclofenac particles having a 0.183 PDI.

### EXAMPLE 3

A control precipitation was performed by rapidly mixing 0.25 ml of 30 mg/ml diclofenac in ethanol with 10 ml aqueous 0.1% (w/v) polyvinyl alcohol 16 at room temperature. The resultant suspension consisted of 7429 nm diclofenac particles crystals with a polydispersity index (PDI) of 0.548. Experimental precipitations were performed by mixing, under the same conditions, 0.25 ml of 30 mg/ml diclofenac in ethanol with 10 ml aqueous 0.05% polyvinyl alcohol 16 and 0.05% sodium deoxycholate. The resultant suspension consisted of 383 nm diclofenac amorphous particles with a PDI of 0.018.

### EXAMPLE 4

A control precipitation was performed by rapidly mixing 0.25 ml of 30 mg/ml diclofenac in ethanol with 10 ml aqueous 0.1% (w/v) Lipoid S-45 at room temperature resulting in a suspension of 2564 nm diclofenac crystalline particles with a polydispersity index (PDI) of 0.525. Experimental precipitations were performed by mixing, under the same conditions, 0.25 ml of 30 mg/ml diclofenac in ethanol with 10 ml aqueous 0.05% Lipoid S-45 and 0.05% sodium deoxycholate. The resultant suspension consisted of 453 nm (PDI=0.197) amorphous diclofenac particles.

### EXAMPLE 5

A control precipitation was performed by rapidly mixing 0.25 ml of 30 mg/ml ethanolic diclofenac with 10 ml aqueous 0.1% sodium laurel sulfate at room temperature. The resultant suspension consisted of 5.5 μm crystals of diclofenac. Experimental precipitations were performed by mixing, under the same conditions, 0.25 ml of 30 mg/ml ethanolic diclofenac with 10 ml aqueous 0.05% sodium laurel sulfate and 0.05% sodium deoxycholate. The resultant suspension consisted of 390 nm (PDI=0.183) amorphous diclofenac particles.

### EXAMPLE 6

A control precipitation was performed by rapidly mixing 0.25 ml of 30 mg/ml acyclovir in ethanol with 10 ml aqueous 0.1% sodium lauryl sulfate at room temperature, resulting in a suspension of 5542 nm (PDI=2.013) acyclovir crystalline particles. Experimental precipitations were performed by mixing, under the same conditions, 0.25 ml of 30 mg/ml ethanolic acyclovir with 10 ml aqueous 0.05% sodium lauryl sulfate and 0.05% sodium deoxycholate, resulting in a suspension of 390 nm (PDI=0.183) amorphous acyclovir particles.

### EXAMPLE 7

A control precipitation was performed by rapidly mixing 0.25 ml of 30 mg/ml ethanolic albendazole with 10 ml aqueous 0.1% polyvinylpyrrolidone (K-17) at room temperature. The resultant suspension consisted of 1830 nm (PDI=0.017) albendazole particles. Experimental precipitations were performed by mixing, under the same conditions, 0.25 ml of 30 mg/ml ethanolic albendazole with 10 ml aqueous 0.05% polyvinyl pyrrolidone (K-17) and 0.05% sodium deoxycholate. The resultant suspension consisted of 174 nm (PDI=0.160) amorphous albendazole particles.

### EXAMPLE 8

A control precipitation was performed by rapidly mixing 0.25 ml of 30 mg/ml acyclovir in dimethyl sulfoxide with 10 ml aqueous 1% polyethylene glycol 300 (PEG 300 MW). The resulting suspension contains crystalline particles 2015 nanometers in diameter with at least half of the product precipitated as (107±22) μm aggregates. However, using an anti-solvent containing 0.5% PEG and 0.5% DOC results in a colloidal suspension of amorphous acyclovir particles of 887 nanometers having a PDI of 0.757.

### EXAMPLE 9

A control precipitation was performed by rapidly mixing 0.25 ml of 100 mg/ml acyclovir in DMSO with 10 ml 0.1% PEG 300 in 50% ethanol at room temperature. The resultant suspension consisted of 706 nm (PDI=0.117) crystalline particles of acyclovir. Experimental precipitations were performed by mixing, under the same conditions, 0.25 ml of 100 mg/ml acyclovir in DMSO with 10 ml 0.05% PEG 300 and 0.05% DOC in 50% ethanol. The resultant suspension consisted of 278 nm (PDI=0.086) acyclovir particles.

### EXAMPLE 10

A control precipitation was performed by rapidly mixing 0.25 ml of 30 mg/ml albendazole in DMSO with 10 ml aqueous 0.10% polymer at room temperature. The resultant suspension consisted of 2 μm crystals of albendazole. Controlled precipitations were performed by rapidly mixing, under the same conditions, 0.25 ml of 30 mg/ml albendazole in DMSO with 10 ml aqueous 0.05% polymer and 0.05% bile acid compounds. The resultant suspensions of albendazole particles are as follows: sodium deoxycholate—105 nm (PDI=0.116); sodium taurodeoxycholate—112 nm (PDI=0.220); sodium cholate—135 nm (PDI=0.148) and sodium taurocholate—105 nm (PDI=0.184). Bile acid compounds yield nanoparticle suspensions that are less than 200 nm with PDI's below 0.20.

### EXAMPLE 11

A control precipitation was performed by rapidly mixing 0.25 ml of an ethanolic solution of a cyclosporine with 10 ml aqueous 0.1% polyvinyl alcohol at room temperature. The resultant suspension consisted of tufts of 1-30 μm crystals. Experimental precipitations were performed by mixing, under the same conditions, 0.25 ml of ethanolic drug solution with 10 ml aqueous 0.05% polyvinyl alcohol and 0.05% sodium deoxycholate. The resultant suspension consisted of 316 nm (PDI=0.115) drug particles.

### EXAMPLE 12

A control precipitation was performed by rapidly mixing 0.25 ml of 30 mg/ml ethanolic diclofenac with 10 ml aqueous 0.1% sodium laurel sulfate at room temperature. The resultant suspension consisted of 5.5 μm crystals of diclofenac. Experimental precipitations were performed by mixing, under the same conditions, 0.25 ml of 30 mg/ml ethanolic diclofenac with 10 ml aqueous 0.05% sodium laurel sulfate and 0.05% sodium deoxycholate. The resultant suspension consisted of 390 nm (PDI=0.183) diclofenac particles.

### EXAMPLE 13

A control precipitation was performed by rapidly mixing 0.25 ml of 50 mg/ml ethanolic cyclosporine with 10 ml aqueous 1.0% Tween 40 at room temperature. The resultant suspension consisted of 327nm (PDI=0.226) particles of cyclosporine. Experimental precipitations were performed by mixing, under the same conditions, 0.25 ml of 50 mg/ml ethanolic cyclosporine with 10 ml aqueous 0.5% Tween 40 and 0.5% sodium deoxycholate. The resultant suspension consisted of 177 nm (PDI=0.126) cyclosporine particles.

This invention has industrial applicability in providing aqueous dispersions of stable nanoparticles and methods for preparation thereof. The inventive nanoparticles and dispersions have medical and non-medical uses.

It is, therefore, apparent that there has been provided, in accordance with the present invention, aqueous dispersions of stable nanoparticles and methods for preparation thereof. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A composition comprising nanoparticles, the nanoparticles comprising at least one aqueous-insoluble compound and at least one bile acid compound, where the at least one aqueous-insoluble compound represents at least 76% of the total weight of the combination of the at least one aqueous-insoluble compound plus the at least one bile acid compound in the nanoparticles.

2. The composition of claim 1, where the at least one bile acid compound represents less than 10% of the total weight of the combination of the at least one aqueous-insoluble compound plus the at least one bile acid compound in the nanoparticles.

3. The composition of claim 2, further comprising at least one surfactant compound.

4. The composition of claim 3, where the at least one surfactant compound represents less than 20% of the total weight of the combination of the at least one aqueous-insoluble compound plus the at least one bile acid compound plus the at least one surfactant compound in the nanoparticles.

5. The composition of claim 3, where the at least one surfactant compound is a non-cationic surfactant compound.

6. The composition of claim 3, where the combined weights of the at least one aqueous-insoluble compound plus the at least one bile acid compound plus the at least one surfactant represent at least 90% of the weight of the nanoparticles in the composition.

7. The composition of claim 1, where the at least one aqueous-insoluble compound is a pharmaceutically useful compound selected from the group consisting of a therapeutic and/or diagnostic compound and a contrast agent.

8. The composition of claim 7, where the at least one pharmaceutically useful compound is a therapeutic and/or diagnostic compound.

9. The composition of claim 8, where the therapeutic and/or diagnostic compound is selected from the group consisting of analgesics, anti-inflammatory agents, antihelminthics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators and xanthines.

10. The composition of claim 1, where the composition has a polydispersity index (“PDI”) selected from the group consisting of a PDI of less than about 1.0, 0.8, 0.6, and 0.4.

11. The composition of claim 10, where the composition has a PDI of less than about 0.4.

12. The composition of claim 1, where the nanoparticles have a mean diameter selected from the group consisting of a mean diameter of less than about 10 μm, 1 μm, 0.5 μm, and 0.2 μm.

13. The composition of claim 12, where the nanoparticles have a mean diameter of less than about 0.2 μ.

14. The composition of claim 1, where the composition has a PDI of less than about 0.4, and the nanoparticles have a mean diameter of less than about 1 μm.

15. The composition of claim 1, where the nanoparticles comprise an aqueous-insoluble compound in a crystalline form, a non-crystalline form, or a combination of crystalline and non-crystalline forms.

16. The composition of claim 15, where the nanoparticles comprise an aqueous-insoluble compound in substantially non-crystalline form.

17. The composition of claim 1, where the at least one bile acid compound is a steroid acid, or salt thereof, including cholic acid, taurocholic acid, glycocholic acid, lithocholic acid, chenodeoxycholic acid, deoxycholic acid, glycodeoxycholic acid, taurodeoxycholic acid, derivatives thereof, and mixtures thereof.

18. A pharmaceutical formulation comprising the composition of claim 1.

19. The formulation of claim 18, where the nanoparticles are dispersed in a tablet, capsule, ointment, cream, film or lyophilized powder/formulation.

20. A method of treating a mammal comprising administering to a mammal an effective amount of the formulation of claim 19.

21-22. (canceled)

23. A method of forming nanoparticles, comprising combining a solution of at least one aqueous-insoluble compound in a first solvent with a miscible precipitation solution comprising at least one bile acid compound so as to form nanoparticles comprising the at least one aqueous-insoluble compound.

24. The method of claim 23, where the miscible precipitation solution further comprises at least one surfactant compound.

25. The composition of claim 24, where the at least one surfactant compound is a non-cationic surfactant compound.
